(19) United States
(12) Patent Application Publication
(10) Pub. No.: US 2010/0204455 A1
(43) Pub. Date: Aug. 12, 2010

(54) Antibody Purification Process By Precipitation

(75) Inventors: David Paul Gervais, Kent (GB); Katherine Anne Pfeiffer, Berkeley, CA (US)

Correspondence Address: PFIZER INC.;PATENT DEPARTMENT, Bld 114 M/S 9114, EASTERN POINT ROAD, GROTON, CT, 06340, US

(73) Assignee: Pfizer Limited

(21) Appl. No.: 12/670,733

(22) PCT Filed: Jul. 18, 2008
(86) PCT No.: PCT/IB08/01882

§ 371 Date: Jan. 26, 2010

Related U.S. Application Data
(60) Provisional application No. 60/952,258, filed on Jul. 27, 2007.

(51) Int. Cl. C07K 16/18; C07K 16/00

(52) U.S. Cl. 530/388.1; 530/387.1

(57) ABSTRACT

The present invention relates to a method of purification of antibodies. An object of the present invention is to provide a method for the isolation of antibodies from a solution containing one or more antibodies, comprising the steps of precipitating the antibody and washing the solid precipitate with washing buffer. Preferably, the antibody is precipitated by using a PEG solution or sodium phosphate.

### FIELD OF THE INVENTION

The present invention relates to a method of purification of antibodies. An object of the present invention is to provide a method for the isolation of antibodies from a solution containing one or more antibodies, comprising the steps of precipitating the antibody and washing the solid precipitate with washing buffer. Preferably, the antibody is precipitated by using a PEG solution or sodium phosphate.

### BACKGROUND OF THE INVENTION

Proteins have become commercially important as drugs that are also generally called “biologicals”. One of the greatest challenges is the development of cost effective and efficient processes for purification of proteins on a commercial scale. While many methods are now available for large-scale preparation of proteins, crude products, such as body fluids or cell harvests, contain not only the desired product but also impurities, which are difficult to separate from the desired product. Moreover, biological sources of proteins usually contain complex mixtures of materials.

Biological sources such as cell culture conditioned media from cells expressing a desired protein product may contain less impurities, in particular if the cells are grown in serum-free medium. However, the health authorities request high standards of purity for proteins intended for human administration. In addition, many purification methods may contain steps requiring application of low or high pH, high salt concentrations or other extreme conditions that may jeopardize the biological activity of a given protein.

Thus, for any protein it is a challenge to establish an efficient purification process allowing for sufficient purity while retaining the biological activity of the protein.

Protein purification generally comprises at least three phases or steps, namely a capture step, in which the desired protein is separated from other components present in the fluid such as DNA or RNA, ideally also resulting in a preliminary purification, an intermediate step, in which proteins are isolated from contaminants similar in size and/or physical/chemical properties, and finally a polishing step resulting in the high level of purity that is e.g. required from proteins intended for therapeutic administration in human or animals.

Typically, the protein purification steps are based on chromatographic separation of the compounds present in a given fluid. Widely applied chromatographic methods are e.g. gel filtration, ion exchange chromatography, hydrophobic interaction chromatography, affinity chromatography or reverse-phase chromatography.

Antibodies or immunoglobulins are an important class of proteins which form part of the naturally-occurring immune systems of mammals, fish, birds and other animals. The antibodies respond to foreign agents, substances, and viral or bacterial infections and help the immune system to reduce or eliminate the threat posed to the host animal. An antibody is usually directed at a specific substance or infection type (the antigen). The affinity between an antibody and its antigen target is highly specific and very strong.

Antibodies can also be manufactured in vivo or in vitro for a variety of uses. Some of these uses might include diagnostic laboratory testing for a particular substance, virus or bacteria; or for the purposes of administering as a pharmaceutical substance (or vaccine) directed against a specific target.

Antibodies can be produced by a number of methods. One method is to expose a host animal such as mouse or rabbit to an antigen of interest, with the purpose of using the animal's own immune system to produce an antibody to that antigen. The antibody is purified from the animal's own bodily fluids or tissues. Another production method is to make the antibodies by cell culture. It is desirable to make antibodies for human pharmaceutical or vaccine use by the cell culture method.

In both the cell culture and the animal production methods for antibodies, the antibodies are typically present in a mixture with other kinds of proteins, carbohydrates, lipids and other molecules. Therefore, the antibodies must be purified in order to be useful for the intended purpose.

In a laboratory setting as well as an industrial setting, the most common purification method for antibodies involves affinity chromatography. In particular, affinity chromatography using Protein A, Protein G or similar is used. Protein A and Protein G are molecules which have a high specificity for antibodies and bind antibodies strongly and reversibly. The Protein A or Protein G are typically chemically/covalently bound to a inert matrix of resin beads that can be packed into a column, such as agarose or Sepharose. Protein A in particular is widely used in the biotechnology industry to purify antibodies on a commercial scale. An example of commercially-available Protein A chromatography media is the mAb Select media available from GE Healthcare (Pollards Wood, Nightingales Lane, Chalfont St Giles, Buckinghamshire, UK).

In running a Protein A chromatography operation, typically the column is equilibrated in a pH-neutral buffer. Then, the cell-free antibody-containing crude mixture from cell culture or animal fluids is passed through the column. The antibodies bind to the Protein A and are retained on the column, while waste materials and contaminants pass through the column. After product loading, the antibody-containing column can be washed with a pH-neutral buffer and then eluted with an acidic buffer to yield an acidic stream containing the antibodies.

There are several issues associated with the use of Protein A, Protein G, and other affinity chromatography operations. One drawback is cost—the cost of the affinity chromatography resin is often orders of magnitude higher than that for other types of chromatography such as ion exchange. Moreover, the Protein A and Protein G molecules sometimes leach from the resin into the antibody product, and are toxic so additional process steps such as Protein A and G removal process as well as control measures and assays must be put in place to remove and monitor any leachate. A further downside is the maximum load for the antibody on the chromatography resin is often quite low (tens of grams of antibody per litre of resin).

The low antibody binding capacity of these affinity resins may create a bottleneck in manufacturing plants both now and in the future. The amount of antibody produced in cell culture systems is increasing as research and development continues on these processes. Therefore the amount of antibody sent to the Protein A column in manufacturing plants will be increasing in the future. As the maximum Protein A column diameters are now being reached in these plants, the only alternative for plants is either to slow down production or to invest heavily in additional Protein A plant capability, which will require significant capital investment. Therefore, an alternative to affinity chromatography process is highly desirable which would give equivalent or better performance than currently used Protein A chromatography in terms of yield and purity.

A process using other types of chromatography is not very attractive because such a process may not remove the amount of impurities removed by Protein A. In particular are the difficult-to-remove heavy chain and light chains which are components of a fully assembled antibody molecule. Furthermore, all types of chromatography have upper limits of capacity and column size and therefore may not offer the type of scalability required for the process of the future.

As a processing technique for monoclonal antibodies, continuous processing may offer real benefits over batch processing, including a reduced capital expenditure for the production facility. Current purification process techniques for monoclonal antibodies (Protein A chromatography) do not lend themselves to continuous production very easily. In particular, the low antibody binding capacity of these affinity resins results a bottleneck in manufacturing plants.

Therefore, it is desired to further refine the currently used antibody purification process and find a way to translate it into a continuous processing format.

A process has now been found in which Protein A can be eliminated from the antibody purification process and replaced using a precipitation/washing system. The precipitation with polyethylene glycol (PEG) or phosphate buffer forces the antibody and other proteins out of solution and into the solid phase, while other contaminants remain soluble. The precipitate can then be washed with a number of wash steps of different compositions to remove various contaminants, including the heavy and light chain impurities.

PEG has already been used as an alternative to classical chromatographic purification processes. In an aqueous two-phase extraction system which has been disclosed in the literature (Andrews B A, Nielsen S, Asenjo J A, “Partitioning and purification of monoclonal antibodies in aqueous two-phase systems.” Bioseparation 1996; 6(5):303-13).

However, the mechanisms governing the partition of biological materials is still not well understood. It depends on many factors such as the concentration and molecular weight of phase forming polymers, the type and quantity of the salt and the type and concentration of additives (usually inorganic salts). Therefore, it is extremely difficult to find the appropriate aqueous two-phase extraction system for a given protein to be purified from a given source.

Similarly, Brooks and al. (Journal of Immunological Methods, Vol. 155 (1992), pages 129-132) discloses a method for the purification of mouse monoclonal antibodies from hybridoma culture supernatants. The method consists in precipitating the antibodies with PEG 6000, recovering the pellet by centrifugation and finally re precipitating the antibodies from the dissolved pellet by using saturated ammonium sulphate. This method of precipitation provides enriched preparations of immunoglobulin but the low yield and level of purity thus obtained is not suitable for therapeutic use in patients where the highest purity is demanded. Further chromatography classical chromatographic purification processes would be required to reach the appropriate level of purity.

Moreover, in the above Brooks' method, the antibody to be purified is subject to several changes (liquid to precipitate, then dissolution followed by re-precipitation) which may increase the risk of aggregation or truncations and unsuitably affect the structure and the function of the antibody.

Finally, the purification method using PEG still needs to be tested with human antibodies and would require several modifications to be adapted to larger industrial scales for manufacture of therapeutic monoclonal antibodies.

Also because the protein intended for therapeutic use must remain fully functional both in terms of structure (e.g. no aggregation, truncations) and in terms of function, any change may render the process unsuitable for therapeutic use in patients.

Then, an alternative to the above process is highly desirable but which would give equivalent or better performance than currently used Protein A chromatography in terms of yield and purity.

To overcome the above downsides of the methods of the prior art, the method of the invention consists in washing a precipitated antibody solids, keeping the antibody in the solid phase, using various wash buffers, and obtain a highly-purified antibody product at good yield.

### SUMMARY OF THE INVENTION

In a present method for the isolation and/or purification of antibodies, it has now surprisingly been found that precipitate antibody solids can then be washed to remove various contaminants, including the heavy and light chain impurities. The present invention allows washing the precipitated antibody solids herein defined as a precipitate, keeping the antibody in the solid phase, using washing buffers. The method of the invention can be used for the isolation and/or purification of antibodies of different kinds with high efficiency and high performance both in terms of yield and purity. Additionally, the method of the present invention meets the strict and demanding requirements for larger industrial scales for manufacture of therapeutic monoclonal antibodies.

The present invention is based upon the discovery that, in polyethylene glycol/sodium phosphate two-phase systems, most of the monoclonal antibodies partitioned as a solid at the liquid-liquid interface. Then, it was found that there was a separation between the monoclonal antibody in the precipitate and the heavy/light chain contaminants which remained soluble.

Therefore, in a first aspect, the invention relates to a method for the isolation of antibodies from a fluid, comprising the steps of (a) precipitating the antibody using a precipitation solution comprising PEG and sodium phosphate; (b) washing the precipitate from step a) with a wash solution comprising PEG and sodium phosphate in adequate concentrations to keep the antibody in a solid phase.

Generally, the method of the invention allows elimination of more method steps than simply the affinity chromatography method.

A second aspect of the invention relates to a bulk antibody preparation obtainable by a method according to the invention.

A third aspect of the invention relates to an antibody formulation obtainable from the bulk of the invention.

In a further embodiment, the method of the present invention extends to work with other, non-antibody proteins produced by cell culture and their purification.

Definitions and General Techniques

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclature used in connection with, and techniques of, cell and tissue culture, molecular biology, immunology, microbiology, genetics and protein and nucleic acid chemistry and hybridization described herein are those well known and commonly used in the art.

The methods and techniques of the present invention are generally performed according to conventional methods well known in the art and as described in various general and more specific references that are cited and discussed throughout the present specification unless otherwise indicated. See, e.g., Sambrook et al. Molecular Cloning: A Laboratory Manual, 3rd ed., Cold Spring Harbor Laboratory Press, Cold Spring Harbor, N.Y. (2000); Ausubel et al., Short Protocols in Molecular Biology: A Compendium of Methods from Current Protocols in Molecular Biology, Wiley, John & Sons, Inc. (2002); Harlow and Lane Using Antibodies: A Laboratory Manual, Cold Spring Harbor Laboratory Press, Cold Spring Harbor, N.Y. (1998); and Coligan et al., Short Protocols in Protein Science, Wiley, John & Sons, Inc. (2003), the disclosures of which are incorporated herein by reference. Enzymatic reactions and purification techniques are performed according to manufacturer's specifications, as commonly accomplished in the art or as described herein. The nomenclature used in connection with, and the laboratory procedures and techniques of, analytical chemistry, synthetic organic chemistry, and medicinal and pharmaceutical chemistry described herein are those well known and commonly used in the art. Standard techniques are used for chemical syntheses, chemical analyses, pharmaceutical preparation, formulation, delivery, and treatment of patients.

The basic antibody structural unit is known to comprise a tetramer. Each tetramer is composed of two identical pairs of polypeptide chains, each pair having one “light” (about 25 kDa) and one “heavy” chain (about 50-70 kDa). The amino-terminal portion of each chain includes a variable region of about 100 to 120 or more amino acids primarily responsible for antigen recognition. The carboxy-terminal portion of each chain defines a constant region primarily responsible for effector function. Human light chains are classified as kappa and lambda light chains. Heavy chains are classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 3 or more amino acids. See generally, Fundamental Immunology Ch. 7 (Paul, W., ed., 2nd ed. Raven Press, N.Y. (1989)) (incorporated herein by reference in its entirety for all purposes). The variable regions of each heavy/light chain pair (VH and VL) form the antibody binding site. Thus, an intact IgG antibody, for example, has two binding sites. Except in bifunctional or bispecific antibodies, the two binding sites are the same.

The variable regions of the heavy and light chains exhibit the same general structure of relatively conserved framework regions (FR) joined by three hyper variable regions, also called complementarity determining regions or CDRs. The term “variable” refers to the fact that certain portions of the variable domains differ extensively in sequence among antibodies and are used in the binding and specificity of each particular antibody for its particular antigen. The variability, however, is not evenly distributed throughout the variable domains of antibodies, but is concentrated in the CDRs, which are separated by the more highly conserved FRs. The CDRs from the two chains of each pair are aligned by the FRs, enabling binding to a specific epitope. From N-terminal to C-terminal, both light and heavy chains comprise the domains FR1, CDR1, FR2, CDR2, FR3, CDR3 and FR4. The assignment of amino acids to each domain is in accordance with the definitions of Kabat Sequences of Proteins of Immunological Interest (National Institutes of Health, Bethesda, Md. (1987 and 1991)), or Chothia & Lesk J. Mol. Biol. 196:901-917 (1987); Chothia et al. Nature 342:878-883 (1989), the disclosures of which are herein incorporated by reference.

As used herein, the term “antibody” is synonymous with immunoglobulin and is to be understood as commonly known in the art. In particular, the term antibody is not limited by any particular method of producing the antibody. For example, the term antibody includes, without limitation, recombinant antibodies, monoclonal antibodies, and polyclonal antibodies. The antibody employed in the present invention may be any class or subclass of antibody. Furthermore, it may be employed irrespective of the purity of the purification starting materials. Examples include natural human antibodies, humanized and human-type antibodies prepared by genetic recombination, monoclonal antibodies of mice. Humanized and human-type monoclonal antibodies are the most useful from an industrial perspective.

The term “antigen-binding portion” of an antibody (or simply “antibody portion”), as used herein, refers to one or more fragments of an antibody that retain the ability to specifically bind to an antigen. It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR).

Where an “antibody” is referred to herein with respect to the present invention, it should be understood that an antigen-binding portion thereof may also be used. An antigen-binding portion competes with the intact antibody for specific binding. See generally, Fundamental Immunology, Ch. 7 (Paul, W., ed., 2nd ed. Raven Press, N.Y. (1989)) (incorporated by reference in its entirety for all purposes). Antigen-binding portions may be produced by recombinant DNA techniques or by enzymatic or chemical cleavage of intact antibodies. In some embodiments, antigen-binding portions include Fab, Fab′, F(ab′)2, Fd, Fv, dAb, and complementarity determining region (CDR) fragments, single-chain antibodies (scFv), chimeric antibodies, diabodies and polypeptides that contain at least a portion of an antibody that is sufficient to confer specific antigen binding to the polypeptide. In embodiments having one or more binding sites, the binding sites may be identical to one another or may be different.

As used herein, the term “human antibody” means any antibody in which the variable and constant domain sequences are human sequences. The term encompasses antibodies with sequences derived from human genes, but which have been changed, e.g. to decrease possible immunogenicity, increase affinity, eliminate cysteines that might cause undesirable folding, etc. The term also encompasses such antibodies produced recombinantly in non-human cells, which might impart glycosylation not typical of human cells. These antibodies may be prepared in a variety of ways, as described below.

The term “chimeric antibody” as used herein means an antibody that comprises regions from two or more different antibodies, including antibodies from different species.

As used herein, the term “humanized antibody” refers to antibodies of non-human origin, wherein the amino acid residues that are characteristic of antibody sequences of the non-human species are replaced with residues found in the corresponding positions of human antibodies. This “humanization” process is thought to reduce the immunogenicity in humans of the resulting antibody. It will be appreciated that antibodies of non-human origin can be humanized using techniques well known in the art. See, e.g. Winter et al. Immunol. Today 14:43-46 (1993). The antibody of interest may be engineered by recombinant DNA techniques to substitute the CH1, CH2, CH3, hinge domains, and/or the framework domain with the corresponding human sequence. See, e.g. WO 92/02190, and U.S. Pat. Nos. 5,530,101, 5,585,089, 5,693,761, 5,693,792, 5,714,350, and 5,777,085). The term “humanized antibody”, as used herein, includes within its meaning, chimeric human antibodies and CDR-grafted antibodies. Chimeric human antibodies of the invention include the VH and VL of an antibody of a non-human species and the CH and CL domains of a human antibody. The CDR-transplanted antibodies of the invention result from the replacement of CDRs of the VH and VL of a human antibody with those of the VH and VL, respectively, of an antibody of an animal other than a human.

The term “isolated antibody” is an antibody that by virtue of its origin or source of derivation (1) is not associated with naturally associated components that accompany it in its native state or (2) is free of other proteins from the same species.

“In vitro” refers to procedures performed in an artificial environment such as, e.g., without limitation, in a test tube or culture medium.

“In vivo” refers to procedures performed within a living organism such as, without limitation, a mouse, rat or rabbit.

“Polyethylene glycol” (PEG) is a hydrophilic, biocompatible and non-toxic water-soluble polymer of general formula H—(OCH₂CH₂)ₙ—OH, wherein n>4. Its molecular weight varies from 200 to 60,000 Daltons.

An antibody can be prepared by recombinant expression of immunoglobulin light and heavy chain genes in a host cell. For example, to express an antibody recombinantly, a host cell is transfected with one or more recombinant expression vectors carrying DNA fragments encoding the immunoglobulin light and heavy chains of the antibody such that the light and heavy chains are expressed in the host cell and, preferably, secreted into the medium in which the host cells are cultured, from which medium the antibodies can be recovered. Standard recombinant DNA methodologies are used to obtain antibody heavy and light chain genes, to incorporate these genes into recombinant expression vectors and to introduce the vectors into host cells, such as those described in Sambrook, Fritsch and Maniatis (eds), Molecular Cloning; A Laboratory Manual, Second Edition, Cold Spring Harbor, N.Y., (1989), Ausubel, F. M. et al. (eds.) Current Protocols in Molecular Biology, Greene Publishing Associates, (1989) and in U.S. Pat. No. 4,816,397, the disclosures of which are incorporated herein by reference.

The term “bulk antibody preparation” refers to the antibody materials which is intended for use as a component of a biological product. These include materials manufactured by processes such as recombinant DNA or other biotechnology methods and isolation/recovery from natural sources. Particularly, it refers to the antibody product obtainable by the method of the invention and prior to any further purification or formulation steps. In a preferred embodiment, the bulk antibody preparation refers the solid washed precipitate dissolved or not in the reconstitution buffer.

The term “batch of antibody preparation” refers to a specific quantity of bulk antibody preparation produced in a process or series of processes so that its expected to be homogeneous within specified limits, particularly by the method of the invention. In the case of continuous production a batch may correspond to a defined fraction of the production, characterised by its intended homogeneity. The batch size may be defined either by a fixed quantity or the amount produced in a fixed time interval.

The term “antibody formulation” refers to a formulation comprising the antibody obtained or obtainable by the method of the invention and further excipients. The bulk antibody preparation can be formulated according to known methods to prepare pharmaceutically useful compositions, wherein an antibody is combined in a mixture with a pharmaceutically acceptable carrier vehicle. Suitable vehicles and their formulation are described, for example, in REMINGTON'S PHARMACEUTICAL SCIENCES (18th ed., Alfonso R. Gennaro, Ed., Easton, Pa.: Mack Pub. Co., 1990). In order to form a pharmaceutically acceptable composition suitable for effective administration, such compositions will contain an effective amount of one or more of the antibodies of the present invention, together with a suitable amount of carrier vehicle.

Preparations may be suitably formulated to give controlled-release of the active compound. Controlled-release preparations may be achieved through the use of polymers to complex or absorb the antibody. The controlled delivery may be exercised by selecting appropriate macromolecules (for example polyesters, polyamino acids, polyvinyl, pyrrolidone, ethylenevinyl-acetate, methylcellulose, carboxymethylcellulose, or protamine, sulfate) and the concentration of macromolecules as well as the methods of incorporation in order to control release. Another possible method to control the duration of action by controlled release preparations is to incorporate the antibody into particles of a polymeric material such as polyesters, polyamino acids, hydrogels, poly(lactic acid) or ethylene vinylacetate copolymers. Alternatively, instead of incorporating these agents into polymeric particles, it is possible to entrap these materials in microcapsules prepared, for example, by coacervation techniques or by interfacial polymerization, for example, hydroxymethylcellulose or gelatine-microcapsules and poly(methylmethacylate)microcapsules, respectively, or in colloidal drug delivery systems, for example, liposomes, albumin microspheres, microemulsions, nanoparticles, and nanocapsules or in macroemulsions. Such techniques are disclosed in Remington's Pharmaceutical Sciences (1980).

The preparation of the invention may be formulated for parenteral administration by injection, e.g., by bolus injection or continuous infusion. Formulations for injection may be presented in unit dosage form, e.g., in ampoules, or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

### BRIEF DESCRIPTION OF THE DRAWINGS

FIG. 1. Diagram of the baseline precipitation process

FIG. 2. Diagram of the continuous up scale precipitation process

FIG. 3. SDS-PAGE Electrophoresis of Baseline (2 mg/ml) Precipitation Experiment.

FIG. 4. Comparative competitive binding assay (ELISA) for bioactivity

FIG. 5. Non-Reduced SDS-PAGE analysis of ANTI-CTLA4 precipitation experiment.

FIG. 6. Non-Reduced SDS-PAGE analysis of IGF1R precipitation experiment.

### DETAILED DESCRIPTION OF THE INVENTION

The present invention is based upon the discovery that, in polyethylene glycol/sodium phosphate two-phase systems, most of the monoclonal antibodies partitioned as a solid at the liquid-liquid interface. Then, it was found that there was a separation between the monoclonal antibody in the precipitate and the heavy/light chain contaminants which remained soluble. Under dilute solution conditions of PEG and/or sodium phosphate, antibodies are soluble. At adequate concentrations of PEG and/or sodium phosphate, the antibody may become insoluble and exist in the solid phase.

The invention relates to a method for the isolation of antibodies from a fluid, comprising the steps of (a) precipitating the antibody using a precipitation solution comprising PEG and sodium phosphate; (b) washing the precipitate from step a) with a wash solution comprising PEG and sodium phosphate in adequate concentrations to keep the antibody in a solid phase.

In accordance with the present invention, the adequate concentrations of PEG and sodium phosphate in the precipitation or wash solution may be any suitable concentrations to keep the antibody in a solid phase as long as the method provides isolation of antibodies with high efficiency and high performance both in terms of yield and purity. In addition, it is to be understood that the same process performance may be realised by using alternatives to PEG or sodium phosphate for the precipitation and the washes of the solid mAb, including but not limited to: potassium phosphate and other phosphate salts, sodium acetate and other acetate salts, sodium sulphate and other sulphate salts, etc.

The concentrations of PEG and/or phosphate which are required to force the antibody or protein to exist in the solid phase will be dependent on a number of factors including the type of antibody or protein, pH, temperature, the concentrations of other solution components (NaCl, solution salts, other reagents, and impurities). It is to be understood that adequate concentrations of PEG and sodium phosphate in the precipitation or wash solution to keep the antibody in a solid phase is to be interpreted by the skilled artisan in light of the teachings and guidance presented herein, in combination with the knowledge of one of ordinary skill in the art.

In a preferred embodiment of the invention, the fluid is added to the precipitation solution under constant agitation and at a constant flow.

In a further preferred embodiment of the invention, the method further comprises a step of recovering the precipitate from the precipitation step (a) or from the washed precipitate of step (b).

In a preferred embodiment of the present invention, the recovering step comprises trapping the precipitate on at least one filter. It is to be understood that the term “filter” include but is not limited to depth filter or any appropriate filter adapted to trap the solid antibody while the solution or buffer which is discarded.

In a highly preferred embodiment, the recovering step comprises trapping the precipitate on two filters, preferably depth filters, which are used in series. Preferably, the first depth filter has a looser pore structure and the second depth filter has a tighter pore structure. More preferably, the first depth filter has a pore structure between approximately 0.2-1.0 microns, and the second depth filter has a pore structure between approximately 0.1-0.5 microns. One example of acceptable depth filters are the 50SP and 90SP grades available from CUNO Limited (3M Centre, Bracknell, Berkshire, UK). It is also advantageous that the wash solution is run through at least one depth filter.

In a particular embodiment, the precipitate is recovered by centrifugation.

In a specific embodiment of the present method of isolation, the precipitation step is repeated at least twice. In a further specific embodiment, the washing step b) is repeated at least twice. While one precipitation step is preferred, the steps of the method of the invention may be repeated any number of time.

In a preferred embodiment, the precipitate is washed in at least two consecutive washes. In a particular embodiment, six consecutive washes are run. If several washes are run, it is preferred, but not necessary, that the wash solution used in the one of the washing step, preferably the first one, is identical to the precipitation solution.

If several washes are run, it may be advantageous that the washing solution of one of the washing step is identical to the precipitation solution.

In a preferred embodiment, the method of the invention further comprises a step (c) of dissolving the precipitate in a reconstitution buffer. In a highly preferred embodiment, the dissolution step (c) is accomplished by flowing the reconstitution buffer through at least one depth filter.

It is understood that the filter, particularly the depth filter described above in connection with precipitating, washing or dissolving step may be used to recover the solid antibody or precipitate after or during any steps of the method and that several separate filters may be used in each step of the method of the invention.

In a preferred embodiment of the method of the invention, the PEG concentration of the precipitation solution or the PEG concentration of the wash solution is between 20% (w/w) and 50% (w/w), preferably between 25 and 35% (w/w) and more preferably 28% (w/w). In such preferred embodiment, the sodium phosphate concentration of the precipitation solution or of the wash solution is between 25 mM and 200 mM, preferably 100 mM.

In a preferred embodiment of the method of the invention, the PEG concentration of the precipitation solution or the PEG concentration of the wash solution is less than 1% (w/w), preferably 0.3% (w/w). In such preferred embodiment, the sodium phosphate concentration of the solution is between 1M and 3M, preferably 1.5M.

In a further preferred embodiment of the invention, the PEG of the precipitation solution and/or the PEG of the wash solution has a molecular weight between 200 and 10,000 Dalton, preferably between 800 and 3000, preferably 1450 Daltons.

In a preferred embodiment of the method of the invention, the concentration of sodium chloride of the precipitation solution or the wash solution is less than 10% (w/w). In highly preferred embodiments, the concentration of sodium chloride of the solution is 0% (w/w), 2% (w/w) or 4% (w/w).

In a further preferred embodiment, the pH of the precipitation solution and the pH of the wash solution is between 3 and 10, preferably between 4 and 7, more preferably 6.

In a preferred embodiment, the concentration of antibody added to the precipitation solution is between 1 g/l and 8 g/l, preferably 2 g/l or 5.5 g/l.

In an embodiment, the fluid containing the antibodies is a cell culture and the cells are removed from the culture by a variety of methods including but not limited to centrifugation, filtration, cross-flow filtration or a combination thereof.

In a preferred embodiment, the fluid containing the antibodies to be purified is a cell culture harvest. The cells and debris may be removed from the culture harvest by a variety of methods including but not limited to centrifugation, filtration, cross-flow filtration or a combination thereof. Preferably, the fluid is ultrafiltrated and may be combined with a precipitation solution or a wash solution. More preferably, the fluid is a clarified cell culture harvest.

In a highly preferred embodiment of the present invention, the concentration of antibody in the fluid is between 1 and 10 g/l. The initial, pre-precipitation concentration of antibody in fluid may be related to the final purity which can be achieved at the end of the method of the invention.

In a further highly preferred embodiment, ammonium sulphate is not used in any steps of the method of the invention.

A second aspect of the invention relates to a bulk antibody preparation obtained or obtainable by a method according to the invention. In a highly preferred embodiment, the bulk antibody preparation of the invention is free of Prot A. It is understood that the term “free of Prot A” means that Prot A concentrations is below the levels detectable by any means available to the man skilled in the art.

In a preferred embodiment of the preparation of the invention, the antibody is a monoclonal anti-CTLA4 antibody or a monoclonal anti IGF1R antibody.

A preferred anti-CTLA-4 antibody is a human antibody that specifically binds to human CTLA-4. Exemplary human anti-CTLA-4 antibodies are described in detail in International Application No. PCT/US99/30895, published on Jun. 29, 2000 as WO 00/37504, European Patent Appl. No. EP 1262193 A1, published Apr. 12, 2002, and U.S. patent application Ser. No. 09/472,087, now issued as U.S. Pat. No. 6,682,736, to Hanson et al., as well as U.S. patent application Ser. No. 09/948,939, published as US2002/0086014, the entire disclosure of which is hereby incorporated by reference. Such antibodies include, but are not limited to, 3.1.1, 4.1.1, 4.8.1, 4.10.2, 4.13.1, 4.14.3, 6.1.1, 11.2.1, 11.6.1, 11.7.1, 12.3.1.1, and 12.9.1.1, as well as MDX-010. Human antibodies provide a substantial advantage in the treatment methods of the present invention, as they are expected to minimize the immunogenic and allergic responses that are associated with use of non-human antibodies in human patients. Characteristics of useful human anti-CTLA-4 antibodies of the invention are extensively discussed in WO 00/37504, EP 1262193, and U.S. Pat. No. 6,682,736 as well as U.S. Patent Application Publication Nos. US2002/0086014 and US2003/0086930, and the amino and nucleic acid sequences set forth therein are incorporated by reference herein in their entirety. Briefly, the antibodies of the invention include antibodies having amino acid sequences of an antibody such as, but not limited to, antibody 3.1.1, 4.1.1, 4.8.1, 4.10.2, 4.13.1, 4.14.3, 6.1.1, 11.2.1, 11.6.1, 11.7.1, 12.3.1.1, 12.9.1.1, and MDX-010. In a more preferred embodiment, the anti-CTLA-4 antibody is 11.2.1.

A further preferred antibody is an anti-IGF1R antibody which is a human antibody that specifically binds to human IGF1R. Exemplary human anti-IGF1R antibodies are described in detail in International Patent Application No. WO 02/053596, published Jul. i 11, 2002, the entire disclosure of which is hereby incorporated by reference; International Patent Application Nos. WO 05/016967 and WO 05/016970, both: published Feb. 24, 2005; International Patent Application No. WO 03/106621, published Dec. 24, 2003; International Patent Application No. WO 04/083248, published Sep. 30, 2004; International Patent Application No. WO 03/100008, published Dec. 4, 2003; International Patent Publication WO 04/087756, published Oct. 14, 2004; and International Patent Application No WO 05/005635, published Jan. 26, 2005.

Because of their ability to block a tumor cell survival pathway, it is desirable to use such anti IGF-1R antibodies to treat cancer, particularly non-hematological malignancies, in patients to obtain an improved clinical benefit relative to standard cancer treatment regimes alone: hormonal therapy agent. Preferably the antibody is one that specifically binds to human IGF 1R. In a preferred embodiment of the present invention, the anti-IGF-1R antibody has the following properties: (a) a binding affinity for human IGF-1 R of Kd of 8×10-9 or less, and (b) inhibition of binding between human IGF-1 R and IGF-1 with an IC50 of less than 100 nM. In another preferred embodiment of the present invention, the anti-IGF-1 R antibody I comprises (a) a heavy chain comprising the amino acid sequences of CDR-1, CDR-2, and i CDR-3 of an antibody selected from the group consisting of 2.12.1, 2.13.2, 2.14.3, 4.9.2, 4.17.3, and 6.1.1, and (b) a light chain comprising the amino acid sequences of CDR-1, CDR 2, and CDR-3 of an antibody selected from the group consisting of 2.12.1, 2.13.2, 2.14.3, 4.9.2, 4.17.3, and 6.1.1, or (c) sequences having changes from the CDR sequences of an antibody selected from the group consisting of 2.12.1, 2.13.2, 2.14.3, 4.9.2, 4.17.3, and 6.1.1, said sequences being selected from the group consisting of conservative changes, wherein the conservative changes are selected from the group consisting of replacement of nonpolar residues by other nonpolar residues, replacement of polar charged residues by other polar uncharged residues, replacement of polar charged residues by other polar charged residues, and substitution of structurally similar residues; and non-conservative substitutions, wherein i the non-conservative substitutions are selected from the group consisting of substitution of: polar charged residue for polar uncharged residues and substitution of nonpolar residues for polar residues, additions and deletions. In a more preferred embodiment, the anti-IGF-1R antibody is 2.13.2 and 4.9.2 as described in detail in International Patent Application No. WO 02/053596.

A third aspect of the invention relates to an antibody formulation obtained or obtainable from the bulk of the invention.

The term “antibody formulation” refers to a formulation comprising the antibody obtained or obtainable by the method of the invention and further excipients. The bulk antibody preparation can be formulated according to known methods to prepare pharmaceutically useful compositions, wherein an antibody is combined in a mixture with a pharmaceutically acceptable carrier vehicle. Suitable vehicles and their formulation are described, for example, in REMINGTON'S PHARMACEUTICAL SCIENCES (18th ed., Alfonso R. Gennaro, Ed., Easton, Pa.: Mack Pub. Co.,1990). In order to form a pharmaceutically acceptable composition suitable for effective administration, such compositions will contain an effective amount of one or more of the antibodies of the present invention, together with a suitable amount of carrier vehicle.

Preparations may be suitably formulated to give controlled-release of the active compound. Controlled-release preparations may be achieved through the use of polymers to complex or absorb the antibody. The controlled delivery may be exercised by selecting appropriate macromolecules (for example polyesters, polyamino acids, polyvinyl, pyrrolidone, ethylenevinyl-acetate, methylcellulose, carboxymethylcellulose, or protamine, sulfate) and the concentration of macromolecules as well as the methods of incorporation in order to control release. Another possible method to control the duration of action by controlled release preparations is to incorporate the antibody into particles of a polymeric material such as polyesters, polyamino acids, hydrogels, poly(lactic acid) or ethylene vinylacetate copolymers. Alternatively, instead of incorporating these agents into polymeric particles, it is possible to entrap these materials in microcapsules prepared, for example, by coacervation techniques or by interfacial polymerization, for example, hydroxymethylcellulose or gelatine-microcapsules and poly(methylmethacylate)microcapsules, respectively, or in colloidal drug delivery systems, for example, liposomes, albumin microspheres, microemulsions, nanoparticles, and nanocapsules or in macroemulsions. Such techniques are disclosed in Remington's Pharmaceutical Sciences (1980).

The preparation of the invention may be formulated for parenteral administration by injection, e.g., by bolus injection or continuous infusion. Formulations for injection may be presented in unit dosage form, e.g., in ampoules, or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

I) Precipitation and Wash Solutions

Precipitation and wash step can be achieved by either PEG solution or Phosphate solution as defined below.

a) PEG Solution:

The PEG solution comprises water, PEG and sodium phosphate. Solid PEG and sodium phosphate reagents are obtained from Sigma Chemical (Poole, Dorset, UK). In specific embodiment, the PEG molecular weight of the PEG solution is between 200 and 10,000 Daltons. In a specific embodiment, a PEG molecular weight between 800 and 3000 is used and preferably, 1450 Daltons. In a preferred embodiment, the concentration of PEG during the precipitation reaction is between 20 and 50% (w/w), preferably between 25-35% (w/w) and most preferably 28% (w/w).

The concentration of sodium phosphate of the PEG solution is between 25 and 200 mM, preferably 100 mM.

In a particular embodiment, a certain amount of sodium chloride is present in the PEG solution to help with the impurity removal. Particularly, the amount of sodium chloride is less than 10% (w/w), preferably 2% (w/w).

In a specific embodiment, the pH of the PEG solution is controlled between 3 and 10 and preferably between 4 and 7, more preferably 6.

b) Phosphate Solution:

The phosphate solution comprises water, PEG and sodium phosphate. Solid PEG and sodium phosphate reagents are obtained from Sigma Chemical (Poole, Dorset, UK).

In specific embodiment, the PEG molecular weight of the PEG solution is between 200 and 10,000 Daltons. In a specific embodiment, a PEG molecular weight between 800 and 3000 is used and preferably, 1450 Daltons. In a preferred embodiment, the concentration of PEG during the precipitation reaction is less than 1% (w/w), preferably 0.3 (w/w).

The concentration of sodium phosphate of the phosphate solution is between 1 and 3 M, preferably 1.5M.

In a particular embodiment, a certain amount of sodium chloride is present in the PEG solution to help with the impurity removal. Particularly, the amount of sodium chloride is less than 10% (w/w), preferably 4% (w/w), more preferably 0%. Sodium chloride solid reagent was obtained from Sigma Chemical (Dorset, Poole, UK).

In a specific embodiment, the pH of the PEG solution is controlled between 3 and 10 and preferably between 4 and 7, more preferably 6.

II) Baseline Method of the Invention

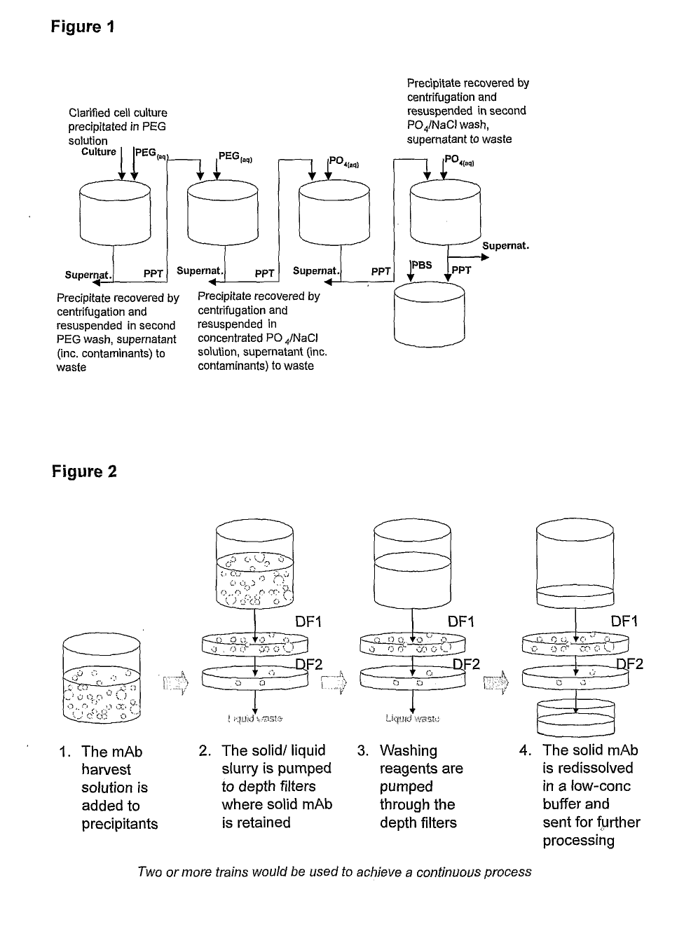

The baseline purification process of the invention is shown in FIG. 1.

In general, the process of capturing and purifying antibodies using precipitation and washing can be split into four steps:
Precipitation of the antibody from a fluid containing antibodies to be purified, e.g. clarified cell culture;
Recovering of the precipitate or solid antibody;
Washing of the solid antibody (precipitate);
Redissolution of the purified antibody.

The specific system of interest (cell culture production system, antibody type, scale of use, antibody intended use etc.) will determine which of the steps are required, repeated and in what sequence.

a) Precipitation

Precipitation step can be achieved by either PEG solution or Phosphate solution.

A fluid containing antibodies, e.g. clarified cell culture, which may or may not be concentrated by a variety of methods including but not limited to ultrafiltration, is added to the PEG or phosphate solution.

In a particular embodiment, the fluid is added to a vessel containing the precipitation solution.

In this solution, the antibody precipitates along with some impurities. Preferably, this solid-liquid slurry is separated by centrifugation or filtration as disclosed in more details in the foregoing description.

In a highly preferred embodiment, the fluid is added to the solution in a well mixed system to achieve the precipitation. More specifically, the precipitation solution is placed on a magnetic stirrer plate and stirred at 300 rpm.

Then, in order to promote the appropriate size precipitate formation, the fluid may be added through a tube e.g. pipette directly into the precipitation solution. In a preferred embodiment, the tube nozzle is submerged. Preferably, the fluid is slowly released, e.g. at 0.5 ml/s, close to the vortex of the stirred solution.

The initial, pre-precipitation concentration of antibody in this combined solution may be related to the final purity which can be achieved at the end of the process. In a specific embodiment, the antibody concentration in this first vessel is between 1 and 8 g/l, preferably 5.5 g/L.

Other alternatives include, but are not limited to, ratio of solid weight to solution volume, the average molecular weight of the PEG, the concentration of the PEG, the pH of the solution, the sodium chloride concentration of the solution, the solid/liquid contact time, the addition flow of the fluid and the temperature.

To allow solid and liquid phases to equilibrate, the contact time between the solid/liquid is preferably controlled and is typically between 0 and 100 minutes and more preferably between 10 and 60 minutes.

It is understood that there may be multiple combinations of these variables which will give acceptable results. Furthermore, it is understood that the optimum values of each variable may vary with the system, the scale and antibody used.

b) Recovering of the Precipitate or Solid Antibody;

In a preferred embodiment, the solid/liquid slurry which results from the precipitation step may be recovered by standard methods such as centrifugation or filtration.

In a further preferred embodiment, the solid/liquid slurry which results from the precipitation step may be recovered by a continuous centrifuge which is capable of discharging the solid product for further processing: This kind of centrifuge capable of solids capture and retention is well known in the art and may be of the Carr™ Separations type or equivalent. The mother liquor or supernatant which is separated contains impurities and may be discarded. This waste stream may also be sent to a recycling unit for re-processing later. The solid which contains the antibody and impurities is retained.

c) Washing of the Precipitate

The solid precipitate, which has been recovered by centrifugation, is retained and is washed.

In a preferred embodiment, the precipitate is re-suspended in the wash solution using standard resuspension methods such as a handheld tissue homogeniser.

In a preferred embodiment, the precipitate is washed in at least two consecutive washes. In a particular embodiment, six consecutive washes are run. The washing step can be repeated as necessary to achieve the desired purity of antibody.

If several washes are run, it is preferred, but not necessary, that the wash solution used in the one of the washing step, preferably the first one, is identical to the precipitation solution.

The variables important to the process include but are not limited, to ratio of solid weight to solution volume, the average molecular weight of the PEG, the concentration of the PEG, the pH of the solution, the sodium chloride concentration of the solution, the solid/liquid contact time, the phosphate concentration of the solution, and the temperature.

The contact time between the solid/liquid is preferably controlled and is typically between 0 and 100 minutes and more preferably between 10 and 60 minutes.

It is understood that there may be multiple combinations of these variables which will give acceptable results. Furthermore, it is understood that the optimum values of each variable may vary with the system, the scale and antibody used.

Multiple PEG and/or phosphate solution washes can be performed if required to achieve the desired antibody purity level. Alternatively, the wash can be skipped altogether if desired.

In a particular embodiment, each wash is followed by a recovering step as previously disclosed.

In a preferred embodiment, one PEG wash and two further phosphate washes are performed.

d) Dissolution of the Solid Antibody

The solid washed precipitate is dissolved in a reconstitution buffer of the type typically used in the art. In a particular embodiment, the precipitate is recovered by centrifugation before dissolution.

Buffers which may be useful for the redissolution step include, but are not limited to, dilute phosphate buffers, acetate buffers, tris buffers, etc. Dilute generally means, but is not restricted to, concentrations in the range of 0-200 mM, preferably 5-100 mM. In a specific embodiment, the pH of the reconstitution buffer is between 4.0 and 7.0.

The volume of buffer used to dissolve the solid may vary and can be chosen based on the concentration of antibody desired.

In a preferred embodiment, the dissolution buffer is a dilute phosphate buffer having a sodium phosphate concentration of 0.1M and pH of 4.9.

Ill) Optimisation to Baseline Process—Continuous Mode—Scale Up

The method of the invention may be run in a batch mode or a continuous mode.

The batch mode may be advantageous for batch-type cell culture production or for continuous perfusion systems. The batch process starts with a cell culture or animal fluid extract which has produced antibodies at a given concentration. The continuous mode may be more suitable for perfusion cell culture systems or very high throughput applications. Such a continuous mode method may involve feeding a continuous stream of clarified cell culture into a reactor or system of reactors in which the precipitation and washing of precipitate takes place.

In a preferred continuous mode of the method of the invention, the solid/liquid slurry which results from the precipitation step may be recovered by a continuous centrifuge which is capable of discharging the solid product for further processing. This kind of centrifuge capable of solids capture and retention is well known in the art and may be of the Carr™ Separations type or equivalent. The mother liquor or supernatant which is separated contains impurities and may be discarded. This waste stream may also be sent to a recycling unit for re-processing later. The solid which contains the antibody and impurities is retained.

In a specific embodiment of the invention, for running an antibody purification process in a continuous fashion, particularly for use with a perfusion bioreactor in a smaller-footprint manufacturing facility, the method of the invention has been developed, which involves recovering the precipitate by trapping it on at least one depth filter and flowing the wash solution through the filter and past the trapped solid antibody.

At the end of the wash step, the re-dissolution of the solid antibody can be accomplished by flowing the reconstitution buffer through a depth filter. In a preferred embodiment, the depth filter setup is first equilibrated with a phosphate solution.

A diagram of the continuous process of the invention is depicted in FIG. 2.

In a preferred embodiment and in order to maximize the yield of the method of the invention, the recovering step consists in trapping the precipitate on two depth filters used in series.

In a specific embodiment, the first depth filter has a looser pore structure and the second depth filter has a tighter pore structure. In a more particular embodiment, the first depth filter has a pore structure of between approximately 0.2-1.0 microns, and the second depth filter has a pore structure of between approximately 0.1-0.5 microns.

In a particular embodiment, the right particle size must be generated the precipitation step in order to facilitate trapping by the depth filters. This may be achieved by first adding the precipitation solution (PEG or phosphate solution) to the precipitation vessel, agitating this mixture to ensure a vortex, and slowly adding the fluid containing the antibody using a pipe with the tip submerged. This ensures excellent mixing and therefore reproducible generation of precipitate or floc size.

IV) Further Purifications

The re-dissolved, purified antibody may be processed further in other downstream purification steps, to achieve the final purity desired. Such further processing steps may include ion-exchange chromatography (cation exchange or anion exchange chromatography), ultrafiltration, diafiltration, viral/Nanofiltration, etc. Anion-exchange chromatography can be conducted with chromatographic resins such as DEAE (diethyl amino ethyl) or Q (quaternary ammonium) and is useful for removal of contaminants such as residual DNA and endotoxins. Cation-exchange chromatography can be conducted with chromatographic resins such as SP (sulfopropyl) and others, and is useful for removing a range of product contaminants such as DNA, host cell proteins, and others. Ion-exchange chromatography resins are available from a range of suppliers such as GE Healthcare (Buckinghamshire, UK). Viral filtration or Nanofiltration is conducted with the use of viral filters available from a range of suppliers (Pall Limited, Portsmouth UK or Asahi Kasei, Japan) and is very useful for the removal or reduction of virus contamination. Such processing steps are well-known in the art, see Janson J C and Ryden L, “Protein Purification”, Wiley and Sons (New York) 1998, Ladisch M R, “Bioseparations Engineering: Principles, Practice and Economics” Wiley InterScience (New York) 2001, or Scopes R K, “Protein Purification: Principles and Practice”, Springer-Verlag (New York) 1994.

The foregoing description of the specific embodiments will be understood that it is capable of further modifications. This application is intended to cover any variations, uses or adaptations of the invention following, in general, the principles of the invention and including such departures from the present disclosure as come within known or customary practice within the art to which the invention pertains and as may be applied to the essential features set forth as follows in the scope of the appended claims.

The foregoing description of the specific embodiments of the invention will so fully reveal the general nature of the invention that others can, by applying knowledge within the skill of the art (including the contents of the references cited herein), readily modify and/or adapt for various application such specific embodiments, without undue experimentation, without departing from the general concept of the present invention. Therefore, such adaptations and modifications are intended to be within the meaning range of equivalents of the disclosed embodiments, based on the teaching and guidance presented herein.

### Preparations and Examples

### Example 1

### Baseline Lab Process

The baseline process has been run several times at laboratory scale and is shown in FIG. 1. Clarified, concentrated anti-CTLA-4 cell culture was used. anti-CTLA-4, 11.2.1 is an IgG2 antibody produced by Pfizer using recombinant DNA technology and cell culture. The cell line used to make 11.2.1 is an NSO mouse myeloma cell line.

a) 1^{st} Run

The precipitation step was done with a system volume of 20 mL. Clarified cell culture solution was concentrated using ultrafiltration (50 kDa molecular weight cut-off) to an antibody concentration of 7.8 g/L (measured by HPLC).

A PEG system was created by the addition of 5.2 ml of the above sample to the following components:

11.2 mL of 50% w/w polyethylene glycol (PEG), stock solution, molecular weight 1450, (Sigma cat. no. P5402-500 g), 1 mL of 2M sodium phosphate stock solution (Acros Organics, CAS no. 10049-21-5), 2 mL of NaCl 20% w/w stock solution (Fisher cat. no. S/3120/60), and 0.65 mL deionised water, resulting in a system volume of 20 ml with an antibody concentration of 2 g/L.

The final concentrations in the 20 ml system volume were 28% w/w PEG-1450, 0.1M phosphate, 2% w/w NaCl, a nominal antibody concentration of 2 g/L, and a pH of appx. 6.7.

The system was agitated on an orbital, shaker at approximately 400 rpm for 30 minutes followed by separation of solid phase by centrifugation (5 minutes at 2400 g). The liquid supernatant was removed and discarded, and a fresh PEG solution was added to the precipitate. This PEG solution was identical to the previous precipitation solution with a nominal antibody concentration of approximately 2 g/L, and a pH of approximately 6.6.

This PEG system was agitated, centrifuged, and decanted as previous. The precipitate was retained and the liquid phase discarded.

A wash step was composed of a phosphate solution, and was added to the precipitate collected in the previous step. The phosphate solution was composed of:

15 mL of 2M sodium phosphate, 4 mL of NaCl 20% w/w, 0.12 mL of 50% w/w PEG-1450, and 0.88 mL deionised water, resulting in a system containing

1.5M phosphate pH, 0.3% PEG, 4% NaCl, appx. 2 g/L antibody, and a pH of appx. 5.9.

The system was again agitated, centrifuged, and supernatant decanted as previously. A final phosphate solution identical to the previous was added, and the system again agitated, centrifuged for 15 minutes at appx. 2400×g, and liquid decanted.

The remaining precipitate w as dissolved to a volume of 10 mL in a 0.1 M sodium phosphate buffer, pH 4.9.

Results

The final re-dissolved precipitate was assayed and the following results generated:

TABLE 1
Analytical results of sample purified by in Example 1.

| | Starting material | Material after precipitation |
|---|---|---|
| Yield | | 91% |
| Host cell protein | Typical between 1e6 | 112 ng/mg |
| (ng/mg) | and 3e6 ng/mg | |
| DNA | Typical between 1e6 | 22 pg/ml |
| | and 9e6 pg/ml | |
| Size exclusion | n.d | 1.245% |
| chromatography | | |
| (% HMMS) | | |
| Potency test | n.d | Compares favourably with |
| (competitive | | reference standard. |
| binding ELISA) | | |
| Purity by SDS-PAGE | | The final precipitate has a |
| Electrophoresis | | purity by SDS-PAGE |
| | | equivalent to the reference |
| | | standard. |

As described in ELISA: Theory and Practice, J R Crowther, Humana Press, New Jersey, USA (1995). A competition ELISA assay is one where two reactants are trying to bind to a third reagent, and the competing reagents are added simultaneously.

b) Additional Runs

In the first two baseline process experiments, a 20 ml system volume was used, 2400 g centrifugation speed, and 2 mg/ml mAb in the 1^{st} PEG precipitation as disclosed above.

In the third baseline experiment, system volume was 100 ml and 10,000 g centrifugation speed. The final re-dissolved precipitates from these experiments were analysed by a variety of methods. A comparison of the results from these precipitation experiments and Protein A chromatography is shown in Table 2.

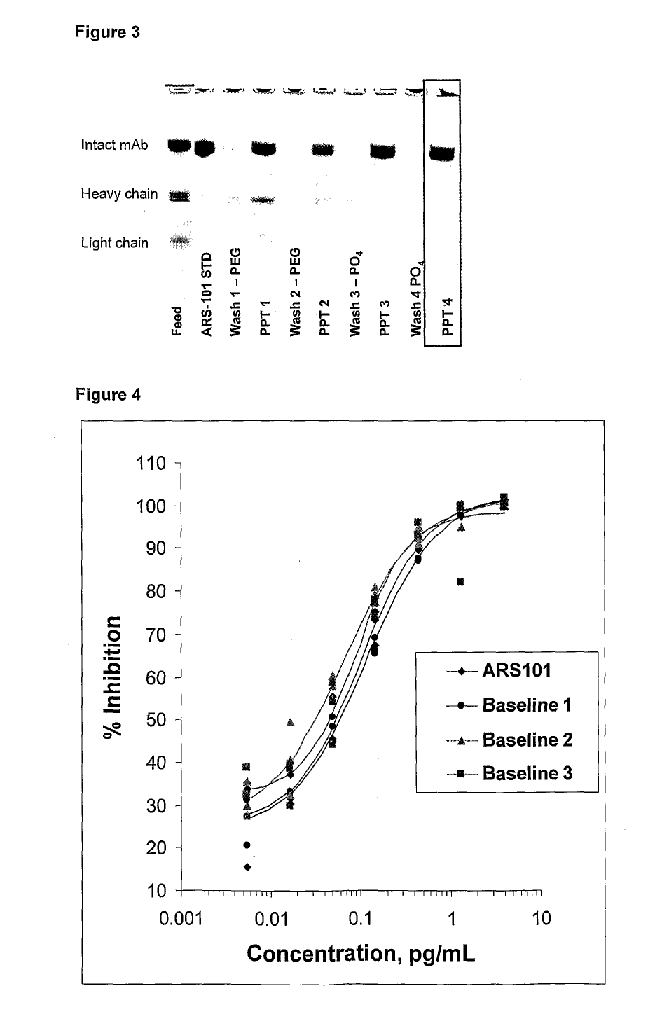

Yield between the two methods is comparable, and yield losses during the precipitation train could be explained by the small (20 ml) scale of these experiments. Little or no carry-over of intact mAb was observed in the washes by SDS-PAGE gel (FIG. 3). Other measures of process impurities including DNA, host cell protein, and residual Protein A were all lower after precipitation when compared with the protein A purified material. The SDS-PAGE profile of the final precipitate (PPT 4) is indistinguishable from the reference standard (ARS101) or from Protein A-purified material.

The reference standard ARS101 was made as part of a fully-purified, standard production run of an anti-CTLA4 antibody. ARS101 was vialed from batch which was the first GMP batch manufactured using the clonal process. It was manufactured at 400 L scale.

The Protein-A purified material refers to anti-CTLA4 antibody which was produced by cell culture and purified through the first chromatography step (Protein A) of the standard production process. The protein-A purified material referred to in this example was manufactured at laboratory scale but any of the known Protein A purification method as mentioned above may be used. Antibody purified by Protein A chromatography would be considered fairly pure, but in a normal manufacturing run would be subjected to further processing steps (chromatography and filtration). To purify a sample using Protein A chromatography, the crude cell-free bioreactor harvest is passed through a column of Protein A media, which had been previously equilibrated with a neutral buffer (pH approximately 7) of phosphate, Tris, or equivalent. The Protein A column will have a maximum capacity for mAb and this may be on the order of 30-40 g mAb/L media. The effluent from this loading phase is discarded, as the mAb binds to the column under these conditions. The column is then washed with a neutral buffer (pH approximately 7) to remove any unbound contaminants. The column may be subjected to further wash steps of varying pH levels, to remove various bound components, before elution with an acidic buffer (pH approximately 3.5). The acidic buffer composition may be low-ionic strength phosphate, acetate, citrate or Tris, or other buffering compounds. The mAb elutes from the column in the acidic buffer and may be taken on for further processing. The column may then be regenerated using a variety of different buffers, to ready the column for subsequent processing cycles.

In addition, precipitated mAb from all three baseline experiments was tested in the competitive binding assay (ELISA) for bioactivity (FIG. 4). This check was necessary to ensure that since the protein had undergone a phase change, no alteration was made to its structure or conformation that would affect the activity. In FIG. 4, the bioassay results showed that the activity of the precipitated mAb was indistinguishable from that of the reference standard (ARS101).

TABLE 2
Comparison of Analytical Results for Precipitation Process with typical
Analytical Results post-Protein A Chromatography.

| | Post- | Post- |
|---|---|---|
| | ProtA | Precipitation |
| | Step | Step |
| Yield | >90% | 83% (n = 3) |
| Host Cell | 316 | 64 (n = 2) |
| Protein (ng/mg) | | |
| DNA (pg/mg) | 1230 | 27 (n = 2) |
| Aggregation by | 0.76% | 1.24% (n = 3) |
| SEC (% HMMS) | | |
| Leached ProtA | 18 | N/A |
| Content (ng/mg) | | |

### Example 2

### Larger Scale Process Conditions

The second example illustrates a process with a nominal antibody concentration of appx 5.5 g/L, and a wash phosphate solution at pH 5.0

Industrial application of this method of antibody purification is more feasible if acceptable purity can be achieved with a higher nominal system concentration of antibody, and thus reduced amounts of buffer materials and shorter run time. High purity at high antibody concentration can be achieved by the lowering the pH of the phosphate wash solution to 5.0, rather than 6.0 as used in the first example.

Clarified cell culture solution was further concentrated to an antibody concentration of 13.0 g/L (HPLC). The initial precipitation was done in a total volume of 160 mL.

Precipitation System Composition is as Follows:

67.1 mL concentrated cell culture solution, 68.9 mL 65% w/w PEG-1450, 16 mL 20% w/w NaCl, and 8.0 mL 2M sodium phosphate buffer pH 6.0,

resulting in a PEG solution containing: nominally 5.43 g/l antibody, 28% w/w PEG-1450, 2% w/w NaCl, 0.1M phosphate, and having a pH of 6.8.

The system was agitated on an orbital shaker for 10 minutes at appx. 300 rpm; 80 mL of the homogenised suspension was removed and centrifuged 10 minutes at 10,000 g to remove the precipitate from suspension. The supernatant was decanted and discarded; the precipitate was re-suspended using a handheld tissue homogeniser (IKA Labrotechnik Ultra Turrax T8, Germany) in a fresh PEG solution, with system composition as follows:

44.8 mL 50% w/w PEG-1450, 8 mL 20% w/w NaCl, 4 mL 2M sodium phosphate buffer pH 6.0, and 5.8 mL deionised water; resulting in a solution containing: nominally appx 5.4 g/L antibody, 28% w/w PEG-1450, 2% w/w NaCl, 0.1M phosphate, and having a pH of approximately. 6.6.

After re-suspension of the precipitate in the fresh PEG solution, 20 mL of the slurry was removed and centrifuged, the supernatant was discarded, and the precipitate was collected as previous. This precipitate were re-suspended in a 20 mL wash phosphate solution, composed of the following:

15 mL 2M phosphate buffer pH 5.0, 4 mL 20% w/w NaCl, and 1 mL of deionised water; resulting in a wash solution containing: nominally approximately. 5.4 g/L antibody, 1.5M phosphate, 4% w/w NaCl, and a small amount of PEG (present due to the residual wash PEG solution in the wet precipitate pellet), and having a pH of approximately 5.1.

After re-suspension, the slurry was agitated, centrifuged, supernatant discarded, and precipitate recovered as previously.

The precipitate was re-suspended, agitated, and centrifuged once more in a second wash phosphate solution nearly identical to that described above, the only difference being the addition of 0.12 mL of 50% w/w PEG-1450, bringing the PEG concentration in the wash to 0.3% w/w. Water was reduced to 0.88 mL so that the total system volume remained 20 mL.

The final precipitate collected after the centrifugation of the second and final phosphate wash was dissolved to a volume of 25 mL in 0.1M sodium phosphate buffer, pH 4.9. Using an homogeniser, the precipitate dissolved freely and easily in this buffer.

Results

Preferably, in order to purify the antibody sample at a nominal concentration of 5.5 g/L, the pH of the phosphate wash is reduced from 6 in the first example to 5 in Example 2. Purity was verified by SDS-PAGE and was comparable to a reference standard sample of the antibody. Results are presented on FIG. 4. The final yield is 83%.

### Example 3

### “Reverse” Method

The Baseline lab process of example 1 has been run using the same clarified, concentrated CP-anti-CTLA-4, 11.2.1, cell culture. The only difference involves precipitation with a phosphate solution and using phosphate solution first and a PEG solution second for the consecutive washes. The method of example 1 used a PEG solution for the precipitation and first wash, and a phosphate solution for the subsequent final washes. The reverse technique has been shown to be reproducible and a typical yield for this technique is 92%. Consequently, the “reverse” precipitation technique has been shown to work in an equivalent way to the “forward” technique of example 1.

### Example 4

### Continuous Process Using Depth Filters

1) Anti-CTLA4

A sample of anti-CTLA4 monoclonal antibody, 11.2.1, clarified broth with a mAb titre of 8.3 mg/ml was precipitated and purified using the reverse technique and the depth filter model, as follows. The total volume of the precipitation system was 160 ml. The precipitation composition was as follows:

57 ml sample of the clarified broth, 80 ml of 3M Phosphate pH 6.0, 1.0 mL of 50% PEG, and 21.8 ml of DI water. To achieve a system concentration of nominally approximately. 3 g/L antibody, 1.5M phosphate, and a small amount of PEG and having a pH of approximately 6.

To achieve the precipitation, the sample was added to the reagents in a well mixed system. First, the reagent mixture was placed on a magnetic stirrer plate and stirred at 300 rpm. Then, the 57 ml mAb sample was pipetted into the mixture with the pipette nozzle submerged close to the vortex. A white precipitate formed in this mixture.

The depth filter setup (two 27 cm² depth filters in series) was first equilibrated with 200 mL of 3M Sodium phosphate pH 6.0. A total of ˜185 mL was collected as equilibration filtrate and discarded. Then, the monoclonal solid/liquid mixture was transferred using a peristaltic pump to the depth filters. These depth filters were from 3M Cuno corporation (Bracknell, UK) and were of two different grades. The first filter in the train was a BC0030A50SP filter (50SP grade) and the second filter in the train was a BC0030A90SP filter (90SP grade). The solid mAb was trapped in these depth filters and the liquid filtrate, which was free of solids, was discarded. Then, three separate 160 ml washes of phosphate buffer were passed through the filter train.

Each wash has the following composition: 80 ml 3M sodium phosphate pH 6.0, 1 ml 50% PEG (MW 1450) and 79 ml DI water to achieve a wash concentration of nominally approximately, 1.5M phosphate, and a small amount of PEG and having a pH of approximately 6.

The filtrates from these washes were discarded. Then, three further 160 ml washes of PEG buffer were passed through the filter train.

Each of the PEG washes had the following composition: 90 ml 50% PEG, 5 ml 3M phosphate pH 6.0, 16 ml of 20% NaCl in water and 49 ml DI water; resulting in wash concentration of approximately: 28% w/w PEG-1450, 2% w/w NaCl, 0.1M phosphate, and having a pH of approximately. 6.0.

The filtrates from the PEG washes were also discarded.

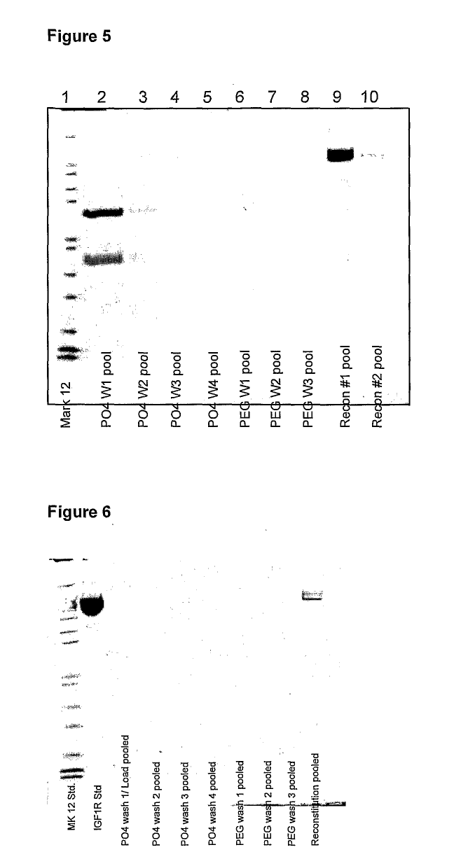

Following the wash steps, the solid mAb was re-dissolved in 372 ml dilute phosphate buffer by passing this buffer through the filter train. The buffer used was 0.1 M sodium phosphate pH 4.9. The filtrate from this re-dissolution was collected. The collected filtrate contained the ANTI-CTLA4 mAb and the overall yield was 100% by Protein A HPLC assay. A further 200 ml dissolution buffer was passed through the filters to confirm removal of all mAb. The product purity of the wash material (lanes 2-8), redissolved mAb in 372 ml (lane 9) and further 200 ml redissolution (lane 10) are shown as a non-reduced SDS-PAGE gel in FIG. 5. The large band at the top of the gel represents the 150 kDa IgG2, and the bands in lanes 2 and 3 at 50 and 25 kDa represent the heavy and light chain impurities, respectively. Consequently the depth filter capture-and-wash technique has been shown to be equivalent to centrifugation for the washing and redissolution of antibody.

2) Anti-IGF1R

A sample of anti-IGF1R monoclonal antibody, 2.13.2, clarified broth with a mAb titre of 1.3 mg/ml was precipitated and purified using the reverse technique and the depth filter model, as follows. The total volume of the precipitation system was 160 ml. The precipitation composition was as follows:

57 ml sample of the clarified broth, 80 ml of 3M Phosphate pH 6.0, 1.0 mL of 50% PEG, and 21.8 ml of DI water; resulting in a solution containing nominally 0.5 g/L antibody, 1.5M phosphate, and a small amount of PEG and having a pH of approximately 6.

To achieve the precipitation, the sample was added to the reagents in a well mixed system. First, the reagent mixture was placed on a magnetic stirrer plate and stirred at 300 rpm. Then, the 57 ml mAb sample was pipetted into the mixture with the pipette nozzle submerged close to the vortex. A white precipitate formed in this mixture.

The depth filter setup (two 27 cm² depth filters in series) was first equilibrated with 200 mL of 3M Sodium phosphate pH 6.0. A total of ˜185 mL was collected as equilibration filtrate and discarded. Then, the monoclonal solid/liquid mixture was transferred using a peristaltic pump to the depth filters. These depth filters were from 3M Cuno corporation (Bracknell, UK) and were of two different grades. The first filter in the train was a BC0030A50SP filter (50SP grade) and the second filter in the train was a BC0030A90SP filter (90SP grade). The solid mAb was trapped in these depth filters and the liquid filtrate, which was free of solids, was discarded. Then, three separate 160 ml washes of phosphate buffer were passed through the filter train.

Each wash had the following composition: 80 ml 3M sodium phosphate pH 6.0, 1 ml 50% PEG (MW 1450) and 79 ml DI water resulting in a solution containing nominally approximately, 1.5M phosphate, and a small amount of PEG and having a pH of approximately 6.

The filtrates from these washes were discarded. Then, three further 160 ml washes of PEG buffer were passed through the filter train.

Each of the PEG washes had the following composition: 90 ml 50% PEG, 5 ml 3M phosphate pH 6.0, 16 ml of 20% NaCl in water and 49 ml DI water; resulting in wash concentration of approximately: 28% w/w PEG-1450, 2% w/w NaCl, 0.1M phosphate, and having a pH of approximately. 6.0.

The filtrates from the PEG washes were also discarded.

Following the wash steps, the solid mAb was re-dissolved in 400 ml dilute phosphate buffer by passing this buffer through the filter train. The buffer used was 0.1 M sodium phosphate pH 4.9. The filtrate from this re-dissolution was collected. The collected filtrate contained the IGF1R mAb and the overall yield was 100% by Protein A HPLC assay. The product purity of the feed material, wash material and redissolved mAb is shown as a non-reduced SDS-PAGE gel in FIG. 6. Consequently the depth filter capture-and-wash technique has been shown to be equivalent to centrifugation for the washing and redissolution of antibody.

1. A method for the isolation of antibodies from a fluid, comprising the steps of:
a) precipitating the antibody using a precipitation solution comprising PEG and sodium phosphate;
b) washing the precipitate from step a) with a wash solution comprising PEG and sodium phosphate in adequate concentrations to keep the antibody in a solid phase.

2. The method of claim 1, wherein the fluid is added to the precipitation solution under constant agitation and at a constant flow.

3. The method of claim 1, further comprising a further step of recovering the precipitate from step (a) or the washed precipitate of step (b).

4. The method of claim 3 wherein the recovering step comprises trapping the precipitate on at least one depth filter.

5. The method of claim 4, wherein two depth filters are used in series.

6. (canceled)

7. The method of claim 5, wherein the first depth filter has a pore structure between approximately 0.2-1.0 microns, and the second depth filter has a pore structure between approximately 0.1-0.5 microns.

8. The method of claim 1 wherein the wash solution is run through at least one depth filter.

9. The method of claim 1 wherein
a) the precipitation step a) is repeated at least twice, and/or
b) the washing step b) is repeated at least twice.

10.-11. (canceled)

12. The method of claim 3 wherein the precipitate is recovered by centrifugation.

13. The method of claim 1 further comprising a step (c) of dissolving the precipitate in a reconstitution buffer.

14. The method of claim 13 wherein the dissolution step (c) is accomplished by flowing the reconstitution buffer through at least one depth filter.

15. The method of claim 1 wherein the PEG concentration of the precipitation solution or the PEG concentration of the wash solution is between 20% (w/w) and 50% (w/w), between 25 and 35% (w/w), or is 28% (w/w).

16.-17. (canceled)

18. The method of claim 15 wherein the sodium phosphate concentration of the precipitation solution or the wash solution is between 25 mM and 200 mM, or the sodium phosphate concentration is 100 mM.

19. (canceled)

20. The method of claim 1 wherein the PEG concentration of the precipitation solution or the PEG concentration of the wash solution is less than 1% (w/w), or is 0.3% (w/w).

21.-23. (canceled)

24. The method of claim 1 wherein the PEG of the precipitation solution and/or the PEG of the wash solution has a molecular weight between 200 and 10,000 Daltons, or between 800 and 3000 Daltons, or is 1450 Daltons.

25. (canceled)

26. The method of claim 1 wherein the concentration of sodium chloride of the precipitation solution is less than 10% (w/w) 4% w/w), 2% (w/w) or 0% (w/w).

27. (canceled)

28. The method of claim 1 wherein the pH of the precipitation solution and the pH of the wash solution is between 3 and 10, between 4 and 7, or the pH is 6.

29.-30. (canceled)

31. The method of claim 1 wherein the concentration of antibody added to the precipitation solution is between 1 g/l and 8 g/l, is 5.5 g/l or is 2 g/l.

32.-37. (canceled)

38. A bulk antibody preparation obtainable by a method according to claim 1.

39. (canceled)

40. The bulk antibody preparation of claim 38 wherein the antibody is a monoclonal anti-CTLA4 antibody or a monoclonal anti IGF1R antibody.

41. (canceled)
